(19) United States
(12) Patent Application Publication
(10) Pub. No.: US 2008/0119806 A1
(43) Pub. Date: May 22, 2008

(54) Extensible Fibers-Method for Their Production and Use

(75) Inventor: Hung Manh Nguyen, Charlotte, NC (US)

Correspondence Address: INVISTA NORTH AMERICA S.A.R.L., THREE LITTLE FALLS CENTRE/1052, 2801 CENTERVILLE ROAD, WILMINGTON, DE, 19808, US

(73) Assignee: Invista North America S.AR.L, Wilmington (DE)

(21) Appl. No.: 11/662,011

(22) PCT Filed: Sep. 9, 2005
(86) PCT No.: PCT/US05/32067

§ 371 Date: Aug. 8, 2007

Related U.S. Application Data
(60) Provisional application No. 60/608,746, filed on Sep. 10, 2004.

(51) Int. Cl. A61F 13/49; D04H 13/00; B29C 65/00

(52) U.S. Cl. 604/366; 442/361; 442/364; 156/60

(57) ABSTRACT

The present invention relates to a bonded nonwoven having extensive fibers for use in articles such as diapers and other hygienic articles, wipes, interlinings and other articles requiring some degree of elasticity. Specifically it relates to bicomponent fibers where one component is an elastic thermoplastic and another component employed as a binder having a lower melting point than the elastic component. On bonding the binder melts to form bonding points exposing the elastic core. The present invention contemplates a bonded nonwoven article or a component thereof, comprising a blend of extensible bicomponent fibers, an absorbent, and optionally low melt binder fibers, or synthetic fibers, or both. The present invention also contemplates a method of producing a nonwoven by mixing the fibers described above, either by the dry laid or wet laid process, and heating the mixture to bond the fibers into a nonwoven article or a component thereof.

### 1) FIELD OF THE INVENTION

The present invention relates to a bonded nonwoven having extensible fibers for use in articles such as diapers and other hygienic articles, wipes, interlinings and other articles requiring some degree of elasticity. Specifically it relates to bicomponent fibers where one component is an elastic thermoplastic and another component employed as a binder having a lower melting point than the elastic component. On bonding the binder melts to form bonding points exposing the elastic core.

### 2) PRIOR ART

In numerous disposable articles, nonwovens require some extensibility. In diapers an extensible nonwoven provides a more comfortable article with reduced leakage. In sanitary articles that consist of wood pulp and super absorbent polymers (SAP) for absorbency, an extensible nonwoven core allows the absorbent to swell to its full potential without being restricted by an inelastic core, like commercially available nonwovens on the market today. In wipes the presence of extensibility makes the product more drapable and less paper-like. In interlinings a more extensible nonwoven again gives a more textile feel to the product.

Elastomers used for elastic films often have an undesirable rubbery feel. When the substances are used in composite nonwovens, the user in contact with the fabric has a rubbery or sticky feeling which is undesirable for direct contact with the skin.

Elastic fabrics usually comprise elastic nonwovens or layers of elastic film. When elastic films are used and the fabric needs to breathe, it is conventional to make holes in the films. These holes may weaken the film and, when stretched, may constitute a site from where tears propagate in the film. Thin films are desirable economically but have limited strength, and this limitation is complicated by the presence of holes.

One method to meet the need for elasticity and for good contact with the skin, is to place a layer of fibrous nonwoven fabric on the elastic layer, producing a composite fabric having improved properties. The nonwoven fabric gives a surface covering the elastomeric layer a soft hand, that is capable of breathing, and suitable for direct contact with the skin. The nonwoven layer also gives additional strength to composite materials. Solutions of this kind are described in U.S. Pat. Nos. 5,921,973; 5,853,881; 5,709,921; 5,681,645; 5,413,849 and 5,334,446.

The composite fabric has to be made in a number of operations with expensive equipment and raw material, including the elastic substances, nonwovens and adhesives.

U.S. Pat. No. 6,541,403 B2 to Billarant et al. discloses the use of a bicomponent fiber having an elastic core surrounded by a non-elastic sheath. The sheath is cut or broken at regular intervals to expose and activate the elastic core.

U.S. Pat. No. 5,352,518 to Muramoto et al. discloses a composite fiber having an elastic core and a sheath that has numerous ridges rising circumferentially and along the length of the fiber. This elastic fiber is not used as a binder fiber in nonwovens, but as a rough elastic fiber for knitted or woven goods.

U.S. Pat. No. 6,225,243 to Austin discloses the use of bicomponent filaments containing an elastic core for use in a spunbond or melt blown nonwoven process.

European Pat. Application 0 454 160 A2 discloses a bicomponent filament comprised of a thermoplastic non-elastomer sheath and a cross-linked polyurethane core. These filaments are used for support type stockings, and no reference is given to their use in nonwoven articles.

Japan Publication 09-031751 discloses a bicomponent fiber comprised of a lower melting non-elastic sheath and an elastic core. The sheath component comprises more than 50% by weight of the fiber and a side-by-side or eccentric configuration is preferred to form crimps in the fiber. These crimped fibers are carded, needle punched and hot air bonded to form an elastic nonwoven article. This publication does not mention a nonwoven having an absorbent.

Japan Publication 2000-282331 discloses a bicomponent fiber comprised of a thin sheath (less than 20% by volume) of a lower melting sheath and an elastic core. This was aimed at protecting the elastic core such that it spun and drew easily to a staple product that could be carded and bonded into a resilient cushion.

Although the dual property of bonding and elasticity in bicomponent fibers with a thermoplastic elastomeric core and a low melting sheath has been disclosed in the Japanese publications, these did not disclose their value in nonwoven structures containing an absorbent such as wood pulp and/or SAP. These publications did not recognize the problem and certainly cannot share in the solution. In addition to the longer fibers required for carded nonwovens there is a need for extensible fibers for use in dry laid and wet laid processes in which short ¼ (0.635 cm) to ½ (1.27 cm) inch fibers are used.

### SUMMARY OF THE INVENTION

The present invention, in its broadest sense, comprises a nonwoven article produced from a blend of an extensible bicomponent fiber, an absorbent and optionally other binder and synthetic fibers. The preferred extensible bicomponent fiber comprises a thermoplastic elastomer as the core, and a thermoplastic sheath having a lower melting point than the core. However a side-by-side bicomponent fiber is also within the scope of the present invention. The present invention, in particular, comprises a nonwoven article or a component thereof, prepared by wet or dry laid processes, utilizing an extensible bicomponent fiber whose sheath melts on heating, thus bonding the nonwoven web and exposes the elastomeric core.

In the broadest sense, the present invention contemplates a bonded nonwoven article comprising a blend of extensible bicomponent fibers, an absorbent, and optionally nonextensible bicomponent fibers, or synthetic fibers, or both.

In the broadest sense the present invention also contemplates a method of producing a nonwoven web by mixing the fibers described above, either by the dry laid or wet laid process, and heating the mixture to bond the fibers into a nonwoven web that can be used to produce a nonwoven article or a component thereof.

In the broadest sense, the present invention also contemplates a diaper that includes as a component a bonded nonwoven web. Such a diaper being an improvement over present day diapers because the extensible fibers allow the diaper to expand as the absorbent swells to its full capacity.

### DETAILED DESCRIPTION OF THE INVENTION

Nonwoven webs of the present invention can be made by either dry laid or wet laid processes. Dry laid webs are made by the airlay, carding, garneting, or random carding processes. Air laid webs are created by introducing the fibers into an air current, which uniformly mixes the fibers and then deposits them on a surface. The carding process separates tufts into individual fibers by combing or raking the fibers into a parallel alignment. Garneting is similar to carding in that the fibers are combed. Thereafter the combed fibers are interlocked to form a web. Multiple webs can be overlapped/stacked to build up a desired weight. Random carding uses centrifugal force to throw fibers into a web with random orientation of the fibers. Again multilayers can be created to obtain the desired web weight. The dry laid components are then bonded together by heating. Wet laid webs are made by a modified papermaking process in which the fibers are suspended in water (or other liquid), the water is separated on a screen to form a web, and the web is dried and bonded by heating.

The webs are bonded using the low melt component (binder) of the bicomponent fibers. The fibers (bicomponent fibers and optionally synthetic fibers) and absorbent can be bonded together to form a web, by thermal means. Thermal bonding in an oven (hot air, radiant or microwave), or heated calendar roll(s), or by ultrasonic energy, melts the low melt component of the bicomponent fibers. The low melt component flows to and coalesces at the junction of the various fibers. Next, the web is cooled thereby solidifying the melted binder. The web now has a sufficient rigid structure to be useful as a component of nonwoven article, or the article itself.

Suitable absorbents are natural or synthetic absorbents. Synthetic absorbents are primarily known as super absorbent polymers (SAP). Natural absorbents are hydrophilic materials such as cellulosic fibers, wood pulp fluff (also known as wood pulp fibers), cotton, cotton linters, and regenerated cellulose fibers such as rayon, or a mixture of these. Preferred is wood pulp fluff, which is both inexpensive and readily available. As used herein, the term “super absorbent polymer” or “SAP” refers to a water-swellable, generally water-insoluble material capable of absorbing at least about 10, desirably about 20, and preferably about 50 times or more its weight in water. The super absorbent polymer may be formed from organic material, which may include natural materials such as agar, pectin, and guar gum, as well as synthetic materials such as synthetic hydrogel polymers. Synthetic hydrogel polymers include, for example, carboxymethyl cellulose, alkali metal salts of polyacrylic acid, polyacrylamides, polyvinyl alcohol, ethylene maleic anhydride copolymers, polyvinyl ethers, hydroxypropyl cellulose, polyvinyl morpholinone, polymers and copolymers of vinyl sulfonic acid, polyacrylates, polyacrylamides, polyvinyl pyridine, and the like. Other suitable polymers include hydrolyzed acrylonitrile grafted starch, acrylic acid grafted starch, and isobutylene maleic anhydride copolymers and mixtures thereof. The hydrogel polymers are preferably lightly crosslinked to render the materials substantially water insoluble. Crosslinking may, for example, be by irradiation or covalent, ionic, van der Waals, or hydrogen bonding. Suitable materials are available from various commercial vendors such as the Dow Chemical Company, Allied Colloid, Inc., and Stockhausen, Inc. The super absorbent polymer may be in the form of particles, flakes, fibers, rods, films or any of a number of geometric forms.

Bicomponent fibers have a low melt component and a high melt component. The fibers can be the side-by-side type or the sheath-core type. Preferable are the sheath-core type. For extensible bicomponent fibers, the high melt component is the elastic thermoplastic, while the low melt component is further described later. For other bicomponent fibers, used as additional binder and/or non-extensible synthetic fiber, the high melt component is the non-extensible synthetic fiber. Again the low melt component is described later. The high melt component (of either the extensible or nonextensible bicomponent fibers) generally comprises from about 35 to about 65 wt. percent of the bicomponent fiber.

The core or high melt component of the extensible bicomponent fiber can be any thermoplastic elastomer. Elastomeric thermoplastic polymers include polyurethane elastomeric materials such as ELASTOLLAN sold by BASF, ESTANE sold by B.F. Goodrich Company, polyester elastomers such as HYTREL sold by E.I. Du Pont De Nemours Company, polyether-ester elastomeric materials such as ARNITEL sold by DSM; and polyether-amide materials such as PEBAX sold by Elf Atochem Company. Of these polyether-ester thermoplastics are preferred. Heterophasic block copolymers, such as those sold by Montel under the trade name CATALLOY are also advantageously employed in the invention. Other elastomeric polymers suitable for this invention are diblock and triblock copolymers based on polystyrene (S) and unsaturated or fully hydrogenated rubber blocks. The rubber blocks can consist of butadiene (B), isoprene (I), or the hydrogenated version, ethylene-butylene (EB). Thus, S-B, S-I, S-EB, as well as S-B-S, S-I-S, and S-EB-S block copolymers can be used. Preferred elastomers of this type include the KRATON polymers sold by Shell Chemical Company and the VECTOR polymers sold by DEXCO. Thermoplastic polyurethanes, which are obtained by reacting a high molecular weight diol and an organic diisocyanate, can also be utilized. The core can be made of materials such as sequenced copolymers, e.g. poly(ethylene-butene), poly(ethylene-hexene) poly(ethylene-propylene) poly(ethylene-octene), poly(styrene-butadiene-styrene), poly(styrene-ethylene and butylene-styrene), poly(styrene-isoprene-styrene), a poly(ester ether oxide), a poly(ether oxide-amide), poly(ethylene-vinyl acetate), poly(ethylene-methylacrylate), poly(ethylene-acrylic acid), poly(ethylene-butyl acrylate) or mixtures thereof.

The sheath polymer for the extensible bicomponent fibers should have a melting point at least about 10° C. below that of the core polymer. It is advantageous for the sheath polymer to rapidly flow during the bonding process so that the unmelted elastic core is exposed. Sheath polymers, as the low melt component, include polyolefin, such as polyethylene (PE), polypropylene (PP), polybutylene or a mixture of these. Suitable polyethylene may be high density polyethylene (HDPE), medium density polyethylene (MDPE), low density polyethylene (LDPE), linear low density polyethylene (LLDPE), ultra low density polyethylene (ULDPE); or a mixture of these. These polyolefins may be produced with either Ziegler-Natta or metallocene catalysts. For adhesion to cellulosic fibers such as wood pulp, it is preferred if the polyolefin sheath contains an adhesion promoter or tackifier.

Adhesion promoters are typically polyolefins grafted with maleic acid or maleic anhydride (MAH), both of which convert to succinic acid or succinic anhydride upon grafting to the polyolefin. The preferred incorporated MAH graft level is 10% by weight (by titration). Also, ethylene-acrylic copolymers, and a combination of this with the grafted polyolefins mentioned are suitable adhesion promoters. Commercially available maleic anhydride grafted polyethylene are known as ASPUN resins from Dow Chemical. Commercially available ethylene-acrylic copolymers are Bynel 2022, Bynel 21E533 and Fusabond MC 190D from DuPont, and the Escor acid terpolymers from ExxonMobil. The ethylene-acrylic copolymer comprises from about 1 to about 20% by weight based on the weight of the polyolefin polymer, and preferably from 5 to 15% by weight. The amount of grafted polyolefin adhesion promoter is such that the weight of incorporated maleic acid or maleic anhydride comprises from about 0.05% to about 2% by weight, and preferably from 0.1 to 1.5% based on the weight of the polyolefin polymer.

Tackifiers include rosin, rosin esters, and terpene based, piperylene based, and hydrocarbon based compounds. Commercially available rosin based tackifiers are known as Foral 85 made by Hercules, Inc.; Permalyn 2085 made by Eastman Chemicals; or Escorez 5400 made by Mobil Exxon Chemical. Commercially available terpene based tackifiers are Zonarez, Zonatac and Nirez from Arizona Chemical Company. Commercially available piperylene based tackifiers are Picotac and Hercotac available from Hercules, Inc. A commercially available hydrocarbon based tackifier is Escorez 5400 from ExxonMobil. The preferred tackifier is rosin ester, and most preferred is a glycerin ester of tall oil rosin. The tackifier preferably comprises from about 0.1 to about 40% by weight of the sheath polymer, and preferably 0.5 to 10%, and most preferably 1 to 5%.

Alternatively, the low melt component or sheath polymer can be a low melting polyester such as polybutylene terephthalate (PBT), or polytrimethylene terephthalate (PTT), a low melting copolyester such as copolymers of PET with comonomers such as suitable diol components selected from 1,4-cyclohexanedimenthanol, 1,2-propanediol, 1,3-propanediol, 1,4-butanediol, 2,2-dimenthyl-1,3-propanediol, 1,6-hexanediol, 1,2-cyclohexanediol, 1,4-cyclohexanediol, 1,2-cyclohexanedimethanol, 1,3-cyclohexanedimethanol, and diols containing one or more oxygen atoms in the chain, e.g., diethylene glycol, triethylene glycol, dipropylene glycol, tripropylene glycol, or mixtures of these; or one or more diacid components other than terephthalic acid, (aliphatic, alicyclic, or aromatic dicarboxylic acids) such as isophthalic acid, 1,4-cyclohexanedicarboxylic acid, 1,3-cyclohexanedicarboxylic acid, succinic acid, glutaric acid, adipic acid, sebacic acid, 1,12-dodecanedioic acid, 2,6-naphthalenedicarboxylic acid, bibenzoic acid, or mixtures of these.

The extensible bicomponent fibers can be of the type in which the low melting portion is adjacent to the high melting portion such as a side-by-side configuration, or a sheath-core configuration where the sheath is the low melting component and the core is the high melting component. The term “sheath” is used to designate the low melting component of the bicomponent fiber. Bicomponent fibers have an average length of from about 3 to about 75 mm, and a denier (decitex (dtex)) of between 1 (1.1) and 10 (11.1).

Nonextensible bicomponent fibers can be used in addition to the extensible bicomponent fiber. The sheath of these bicomponent fibers is selected from the same classes of polymers as noted for the extensible bicomponent fiber. The core or high melt component may be selected from the class of polyolefins, such as polypropylene, and polybutylene; polyesters such as polyethylene terephthalate (PET), polybutylene terephthalate, polyethylene naphthalate, and the like; polyamides such as nylon 6, nylon 66, and the like; polyacrylates such as polymethacrylate, polymethylmethacrylate, and the like; as well as mixtures and copolymers of these. The low melting component, in a nonextensible bicomponent fiber melts at a temperature of at least about 10° C. lower than the high melting portion.

Other synthetic fibers can be used in the preparation of the nonwoven web in addition to the extensible bicomponent fiber, nonextensible bicomponent fiber and absorbent. These fibers can be of any cross-section, for instance round, hollow or multilobal. Suitable synthetic fibers are polyester, polyolefin, nylon, polyacrylates and the like. These fibers are essentially the same as those for the high melt component of the nonextensible bicomponent. Thus an optional nonextensible bicomponent fiber can be a source of both a synthetic fiber, or a low melt binder component, or both. Preferred synthetic fibers are those made from PET.

The webs are made by merely mixing the bicomponent fibers and optional synthetic fibers with the absorbent in fiber form or otherwise, using dry laid or wet laid techniques. The absorbent is mixed with the bicomponent fiber such that the extensible bicomponent fibers comprises from about 5 to about 50 percent by weight of the total web, with the remainder being substantially the absorbent. The optional synthetic fibers can comprise up to half of the extensible bicomponent fibers (i.e. replace some of the extensible bicomponent fibers with synthetic fibers). When it is desirable to produce a web that requires synthetic fibers, it is usually necessary to add other nonextensible bicomponent fibers to act as the low melt binder and the synthetic fiber component. Thus the extensible bicomponent fiber can be the sole bicomponent fiber or mixed with nonextensible bicomponent fibers. In addition conventional synthetic fibers such as polyester can be mixed with the other fibers. The web compositions of the present invention can be layered until their weight is in the range from about 20 to about 500 grams per square meter (gsm), preferably from about 50 to about 250 gsm. Thereafter, the web may be cut into various lengths and widths for end use applications, namely, wipes, fenestration drapes, dental bibs, eye pads, diapers, incontinent pads, sanitary napkins, wound dressing pads, air filters, liquid filters and fabrics such as drapes, bedding, pillows, cushions and other insulating products.

### Test Procedure

The wet and dry strength of the web was measured according to TAPPI test methods T 456 om-87 and T 494 om-88 respectively. The wet strength was measured after an immersion time of 15 sec. The web strength was tested on a 1 inch (2.5 cm)×8 inch (20 cm) strip. The tests were run at 5 inch (12.5 cm) original separation at a cross-head speed of 12 inch (30 cm) per minute. The strength is reported in units of lb./inch (kg/cm).

The basis weight was measured according to TAPPI test method T 410 om-93, and reported in g/m².

### EXAMPLE 1

An extensible bicomponent fiber comprising a core of a thermoplastic elastomer (DSM type EM400, melting point 195° C.) and a sheath of linear low density polyethylene (Dow Chemical Company ASPUN 34, melting point 128° C.) with a core:sheath weight ratio of 65:35 was prepared using the method described in U.S. Pat. No. 5,505,899. The fiber had a denier per filament (dpf) of 4.6 (5.1 dtex) and was cut to a 2 inch (5 cm) length.

### EXAMPLE 2

A second extensible bicomponent fiber was prepared in the same manner as Example 1. In this example the sheath polymer was an amorphous copolyester (broad melting range around 110° C.), and the fiber had a dpf of 4.8 (5.3 dtex) and was cut to a 2″ (5 cm) length.

### EXAMPLE 3

A third extensible bicomponent fiber was prepared in the same manner as Example 1. In this case the polyethylene sheath (melting point 128° C.) was grafted with maleic anhydride (MAH) by melt blending 10 weight % of Dow ASPUN 07 with the ASPUN 34. The dpf was 4.3 (4.7 dtex) and the cut length was ½ inch (12.25 cm).

### EXAMPLE 4

A fourth extensible bicomponent fiber was prepared in the same manner as Example 3 to give a dpf of 4.6 (5.1 dtex) and was cut to ¼ inch (0.635 cm).

### EXAMPLE 5

Carded webs were prepared with 40% standard round cross section polyester staple fiber having a dpf of 3 (3.3 dtex) and a cut length of 1.5 inch (3.75 cm) (INVISTA type 224) and 60% by weight of bicomponent fibers. These include the extensible bicomponent fibers of Examples 1 and 2, and a commercially available bicomponent binder fiber: INVISTA Type 256 which is a 3 dpf (3.3 dtex), 1.5 inch (3.75 cm) cut fiber having the same polyethylene sheath as Example 1 and a polyester core in a core:sheath weight ratio of 50:50.

The carded webs were bonded in an oven at 135° C. for 10 seconds. The webs were cycled between zero and 96% elongation for three cycles, and the total load (lb.) (kg) measured at this 96% elongation on each cycle. The results are set forth in Table 1.

TABLE 1

| | | | | Load |
|---|---|---|---|---|
| Bicomponent | Basis | Load after 1^{st} | Load after 2^{nd} | after 3^{rd} |
| Fiber type | weight g/m² | cycle lb. (kg) | cycle lb. (kg) | cycle lb. (kg) |
| T-256 | 98 | 9.69 (4.4) | 1.75 (0.8) | 0.79 (0.36) |
| Example 2 | 116 | 3.39 (1.54) | 0.5 (0.23) | 0.3 (0.14) |
| Example 1 | 95 | 2 (0.91) | 1.97 (0.86) | 1.95 (0.89) |

This example illustrates that a carded web containing 60% of the extensible fiber with a polyethylene sheath retained its strength after being stretch approximately 100%, whereas the other bicomponent binder fibers lose over 90% of their strength. It is believed from microscopic examination of webs that the copolyester sheath of the extensible bicomponent fiber of Example 2 did not flow sufficiently to expose the elastic core. The extensible bicomponent of Example 1 will have value in nonwoven articles requiring elastic recovery in addition to extensibility, such as, for example, bonded batts and cushions.

### EXAMPLE 6

Wet laid nonwoven webs were prepared with a mixture of bicomponent fibers at a 30 weight % level with wood pulp (Rayonnier Rayocel HF). The extensible bicomponent prepared in Example 3 was used in conjunction with INVISTA Type 105 uncrimped bicomponent 1.5 dpf (1.65 dtex), ¼ inch (0.635 cm) fiber which has the same grafted polyethylene sheath as the Example 3 and a polyester core in a 50:50 weight ratio. The webs were bonded at 175° C. for 30 seconds. The basis weight of the nonwoven webs was 60-65 g/m². The dry and wet web strengths are set forth in Table 2.

TABLE 2

| Bicomponent blend | | Dry | | Wet | |
|---|---|---|---|---|---|
| level, % | | Strength, | | Strength, | |
| Example 3 | T-105 | lb./in.(kg./cm.) | Elongation, % | lb./in.(kg./cm.) | Elongation, % |
| 0 | 30 | 3.25(0.58) | 17 | 1.9(0.34) | 13 |
| 10 | 20 | 2.9(0.52) | 19 | 2(0.36) | 18 |
| 15 | 15 | 2.1(0.37) | 21 | 1.6(0.29) | 22 |
| 20 | 10 | 1.5(0.27) | 24 | 0.8(0.14) | 23 |
| 30 | 0 | 0.9(0.16) | 39 | 0.5(0.09) | 44 |

The use of the extensible bicomponent fiber increases the elongation of the web. This is an important attribute for nonwovens such as wipes, in which a softer hand and the ability to deform the wipe is important. This more extensible web has value as the absorbent core in diapers and feminine hygiene products since it allows the absorbent, especially SAP, to swell to its full capacity.

### EXAMPLE 7

In this example air laid webs were prepared from a blend of bicomponent fibers with 70 weight % wood pulp (Weyco NF401 fluff pulp). The extensible bicomponent fiber of Example 4 was used, together with a commercial 2 dpf (2.2 dtex), ¼ inch (0.635 cm) crimped bicomponent fiber having the same MAH grafted polyethylene sheath as Example 4 and a polyester core in a 50:50 weight ratio (INVISTA T-255). In addition a 3-layer nonwoven web was prepared. The basis weight was 57-60 g/m2 and the webs were bonded at 175° C. for 7 seconds. The dry and wet web strengths for the homogeneous blends are set forth in Table 3, and those for the layer structure in Table 4.

TABLE 3

| Bicomponent blend | | Dry | | Wet | |
|---|---|---|---|---|---|
| level, % | | Strength, | | Strength, | |
| Example 4 | T-255 | lb./in.(kg./cm.) | Elongation, % | lb./in.(kg./cm.) | Elongation, % |
| 0 | 30 | 2(0.36) | 27 | 1.2(0.22) | 26 |
| 10 | 20 | 1.9(0.34) | 31 | 1(0.18) | 30 |
| 20 | 10 | 1.2(0.22) | 36 | 0.8(0.14) | 38 |
| 30 | 0 | 0.6(0.11) | 37 | 0.4(0.07) | 38 |

### The increase in elongation in the air laid nonwoven web is comparable to that seen in Example 6.

TABLE 4

| Top | | | | Bottom | | | | | |
|---|---|---|---|---|---|---|---|---|---|
| Layer | | Middle Layer | | layer | | Dry | | Wet | |
| Ex. | T- | Wood | T- | Ex. | T- | Str. | | Str. | |
| 4, % | 255, % | pulp, % | 255, % | 4, % | 255, % | lb./in.(kg./cm.) | Elong. % | lb./in.(kg./cm.) | Elong. % |
| 10 | — | 70 | 10 | 10 | — | 1.6(0.29) | 37 | 0.7(0.13) | 45 |
| 5 | 10 | 70 | — | 5 | 10 | 2.2(0.39) | 30 | 2.2(0.39) | 36 |
| 10 | 5 | 70 | — | 10 | 5 | 1.4(0.25) | 35 | 1.35(0.24) | 39 |

### The inclusion of the extensible fiber gives a tougher more extensible nonwoven web.

Thus it is apparent that there has been provided, in accordance with the invention, an article that fully satisfied the objects, aims and advantages set forth above. While the invention has been described in conjunction with specific embodiments thereof, it is evident that many alternatives, modifications and variations will be apparent to those skilled in the art in light of the foregoing description. Accordingly, it is intended to embrace all such alternatives, modifications and variations as fall within the spirit and broad scope of the appended claims.

1. A bonded nonwoven article comprising: a blend of an extensible bicomponent fiber, an absorbent, and optionally nonextensible bicomponent fibers, or synthetic fibers, or both, wherein said extensible bicomponent fiber comprises a thermoplastic elastomer and a thermoplastic Polymer having a lower melting point than said elastomer.

2. (canceled)

3. The nonwoven article of claim 1, wherein said bicomponent fiber is a sheath/core fiber wherein said elastomer is the core and the polymer is the sheath.

4. The nonwoven article of claim 1, wherein said absorbent comprises wood pulp.

5. The nonwoven article of claim 1, wherein said absorbent comprises a blend of wood pulp and super absorbent polymer.

6. The nonwoven article of claim 1, wherein said synthetic fibers comprise polyester.

7. The nonwoven article of claim 1, wherein said thermoplastic polymer comprises polyolefin.

8. The nonwoven article of claim 7, wherein said polyolefin is grafted linear low density polyethylene.

9. The nonwoven article of claim 1, wherein said article is a wipe, a component of a diaper, or a component of a feminine hygiene article.

10. A method of producing a bonded nonwoven article comprising, adding to liquid, extensible bicomponent fibers, absorbent fibers, and optionally nonextensible bicomponent fibers, or synthetic fibers, or both; mixing said extensible bicomponent fibers, absorbent fibers, and said optional fibers if any, with said liquid, thereby forming a fiber mixture, and wet laying said fiber mixture by separating said liquid from said fiber mixture to form a web; heating said web sufficiently to bond said web, thereby forming said nonwoven article or a component thereof; wherein said extensible bicomponent fibers comprise a thermoplastic elastomer and a thermoplastic polymer having a lower melting point than said elastomer.

11. (canceled)

12. The method of claim 10, wherein said bicomponent fibers are sheath/core fibers wherein said elastomer is the core and the polymer is the sheath.

13. A method of producing a bonded nonwoven article comprising, mixing extensible bicomponent fibers, absorbent, and optionally nonextensible bicomponent fibers, or synthetic fibers, or both thereby forming a web; heating said web sufficiently to bond said web, thereby forming said nonwoven article or a component thereof; wherein said extensible bicomponent fibers comprise a thermoplastic elastomer and a thermoplastic polymer having a lower melting point than said elastomer.

14. (canceled)

15. The method of claim 14, wherein said bicomponent fibers are sheath/core fibers wherein said elastomer is the core and the polymer is the sheath.

16. A diaper having as a component a bonded nonwoven web, comprising: a blend of an extensible bicomponent fiber, an absorbent, and optionally nonextensible bicomponent fibers, or synthetic fibers, or both to form a web, wherein said extensible bicomponent fiber comprises a thermoplastic elastomer and a thermoplastic polymer having a lower melting point than said elastomer.

17. The diaper of claim 16, wherein said absorbent comprises wood pulp fibers, super absorbent polymer, or both.

18. The nonwoven article of claim 1, wherein said absorbent is about 50 to about 95 wt. % and said extensible bicomponent fibers are about 5 to about 50 wt. %.

19. The method of claim 10, wherein said absorbent fibers are about 50 to about 95 wt. % and said extensible bicomponent fibers are about 5 to about 50 wt. %.

20. The method of claim 13, wherein said absorbent is about 50 to about 95 wt. % and said extensible bicomponent fibers are about 5 to about 50 wt. %.

21. The nonwoven article of claim 2, wherein said thermoplastic elastomer is about 35 to about 65 wt. percent of said extensible fiber.

22. The nonwoven article of claim 1, wherein said thermoplastic elastomer is selected from the group consisting of: polyurethane elastomers, polyester elastomers, polyether-ester elastomers, polyether-amide elastomers, heterophasic block copolymers, diblock copolymers, triblock copolymers, and combinations thereof.

23. The nonwoven article of claim 22, wherein said diblock and triblock copolymers comprise at least one of polystyrene, butadiene, isoprene, or ethylene-butylene.

24. The nonwoven article of claim 1, wherein said thermoplastic elastomer comprises poly(ethylene-butene), poly(ethylene-hexene) poly(ethylene-propylene) poly(ethylene-octene), poly(styrene-butadiene-styrene), poly(styrene-ethylene and butylene-styrene), poly(styrene-isoprene-styrene), a poly(ester ether oxide), a poly(ether oxide-amide), poly(ethylene-vinyl acetate), poly(ethylene-methylacrylate), poly(ethylene-acrylic acid), poly(ethylene-butyl acrylate) and combinations thereof.

25. The method of claim 10, wherein said thermoplastic elastomer is selected from the group consisting of: polyurethane elastomers, polyester elastomers, polyether-ester elastomers, polyether-amide elastomers, heterophasic block copolymers, diblock copolymers, triblock copolymers, and combinations thereof.

26. The method of claim 25, wherein said diblock and triblock copolymers comprise at least one of polystyrene, butadiene, isoprene, or ethylene-butylene.

27. The method of claim 10, wherein said thermoplastic elastomer comprises poly(ethylene-butene), poly(ethylene-hexene) poly(ethylene-propylene) poly(ethylene-octene), poly(styrene-butadiene-styrene), poly(styrene-ethylene and butylene-styrene), poly(styrene-isoprene-styrene), a poly(ester ether oxide), a poly(ether oxide-amide), poly(ethylene-vinyl acetate), poly(ethylene-methylacrylate), poly(ethylene-acrylic acid), poly(ethylene-butyl acrylate) and combinations thereof.

28. The method of claim 13, wherein said thermoplastic elastomer is selected from the group consisting of: polyurethane elastomers, polyester elastomers, polyether-ester elastomers, polyether-amide elastomers, heterophasic block copolymers, diblock copolymers, triblock copolymers, and combinations thereof.

29. The method of claim 13, wherein said diblock and triblock copolymers comprise at least one of polystyrene, butadiene, isoprene, or ethylene-butylene.

30. The method of claim 28, wherein said thermoplastic elastomer comprises poly(ethylene-butene), poly(ethylene-hexene) poly(ethylene-propylene) poly(ethylene-octene), poly(styrene-butadiene-styrene), poly(styrene-ethylene and butylene-styrene), poly(styrene-isoprene-styrene), a poly(ester ether oxide), a poly(ether oxide-amide), poly(ethylene-vinyl acetate), poly(ethylene-methylacrylate), poly(ethylene-acrylic acid), poly(ethylene-butyl acrylate) and combinations thereof.
